(19) United States
(12) Patent Application Publication
(10) Pub. No.: US 2018/0236142 A1
(43) Pub. Date: Aug. 23, 2018

(54) USE OF DUAL MOLD RELEASE AGENTS FOR MEDICAL DEVICE APPLICATIONS

(71) Applicant: SABIC Global Technologies B.V., Bergen op Zoom (NL)

(72) Inventors: Bin SUN, Newburgh, IN (US); James Franklin HOOVER, Evansville, IN (US)

(21) Appl. No.: 15/753,039

(22) PCT Filed: Aug. 30, 2016
(86) PCT No.: PCT/US2016/049470

§ 371 Date: Feb. 15, 2018

Related U.S. Application Data
(60) Provisional application No. 62/212,403, filed on Aug. 31, 2015.

(51) Int. Cl. A61L 31/06; B29C 45/00; A61M 5/31

(52) U.S. Cl. CPC A61L 31/06; B29K 2069/00; A61M 5/3129; B29C 45/0001

(57) ABSTRACT

The disclosure concerns medical device parts having a thickness of about 4 mm or less, where the medical device part is manufactured by an injection molding process utilizing a polycarbonate polymer and from about 0.2 wt % to about 0.5 wt % of pentaerythritol tetrastearate and from about 0.05 wt % to about 0.3 wt % of glycerol monostearate; wherein said pentaerythritol tetrastearate is derived from a biosource.

### CROSS-REFERENCE TO RELATED APPLICATIONS

This application claims priority to U.S. Provisional Patent Application Ser. No. 62/212,403 filed on Aug. 31, 2015, the disclosure of which is incorporated herein in its entirety.

### TECHNICAL FIELD

The disclosure concerns use of dual mold release agents in the formation of polycarbonate-containing medical device parts.

### BACKGROUND

Current process for producing polycarbonate-based medical device parts by injection molding utilize a single mold release agent. Typically, a mold release agent (pentaerythritol tetrastearate or PETS) that is derived from vegetables in the formulation (sometime referred to as “veggie PETS”) is used to comply with the regulations for use of plastics in the healthcare and medical device industries. These processes, however, can be deficient in obtaining clean release from the mold—especially with product having a portion that is 4 mm or less in thickness. There is a need in the art for an improved process.

### SUMMARY

In some aspects, the disclosure concerns medical device parts having a thickness of about 4 mm or less, where the medical device part is manufactured by an injection molding process utilizing a polycarbonate polymer and from about 0.2 wt % to about 0.5 wt % of pentaerythritol tetrastearate and from about 0.05 wt % to about 0.3 wt % of glycerol monostearate; wherein said pentaerythritol tetrastearate is derived from a biosource. In some embodiments, glycerol tristearate may be used in addition to or in place of glycerol monostearate. In some embodiments, the total amount of glycerol monostearate and glycerol tristearate does not exceed 1 wt % based on the weight of the medical device part. In certain aspects, a mixture of stearate esters of glycerol (full or partial esters) may be used. In some embodiments, the total amount of stearate ester mixture does not exceed 1 wt % based on the weight of the medical device part. In yet other embodiments, glycerol tristearate is used as the sole mold release agent. The amount of glycerol tristearate in this latter embodiment does not exceed 1 wt % based on the weight of the medical device part.

In other aspects, the disclosure concerns processes for the manufacture of medical device parts, the processes comprising injection molding polycarbonate polymer and comprising from about 0.2 wt % to about 0.5 wt % of pentaerythritol tetrastearate and from about 0.05 wt % to about 0.3 wt % of glycerol monostearate; wherein said pentaerythritol tetrastearate is derived from a biosource and all wt % values are based on the weight of the medical device part. In some embodiments, glycerol tristearate may be used in addition to or in place of glycerol monostearate. In some embodiments, the total amount of glycerol monostearate and glycerol tristearate does not exceed 1 wt % based on the weight of the medical device part. In yet other embodiments, glycerol tristearate is used as the sole mold release agent. The amount of glycerol tristearate in this latter embodiment does not exceed 1 wt % based on the weight of the medical device part.

### BRIEF DESCRIPTION OF THE DRAWINGS

FIG. 1 presents an illustration of one representative syringe.

### DETAILED DESCRIPTION OF ILLUSTRATIVE EMBODIMENTS

In this disclosure, medical devices and processes for making the devices are described. In some aspects, a combination of mold release additives is utilized to improve production performance. In certain aspects, the medical device part comprises a syringe component such as a syringe housing.

### Polycarbonate (PC)

The terms “polycarbonate” or “polycarbonates” as used herein includes copolycarbonates, homopolycarbonates and (co)polyester carbonates. PC polymers are available commercially from SABIC.

The term polycarbonate can be further defined as compositions have repeating structural units of the formula (1):

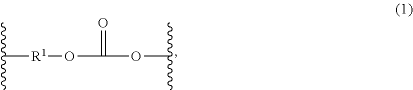

in which at least 60 percent of the total number of R¹ groups are aromatic organic radicals and the balance thereof are aliphatic, alicyclic, or aromatic radicals. In a further aspect, each R¹ is an aromatic organic radical and, more preferably, a radical of the formula (2):

-A¹-Y1-A²- (2),

wherein each of A¹ and A² is a monocyclic divalent aryl radical and Y¹ is a bridging radical having one or two atoms that separate A¹ from A². In various aspects, one atom separates A¹ from A². For example, radicals of this type include, but are not limited to, radicals such as —O—, —S—, —S(O)—, —S(O₂)—, —C(O)—, methylene, cyclohexyl-methylene, 2-[2.2.1]-bicycloheptylidene, ethylidene, isopropylidene, neopentylidene, cyclohexylidene, cyclopentadecylidene, cyclododecylidene, and adamantylidene. The bridging radical Y1 is preferably a hydrocarbon group or a saturated hydrocarbon group such as methylene, cyclohexylidene, or isopropylidene. Polycarbonate materials include materials disclosed and described in U.S. Pat. No. 7,786,246, which is hereby incorporated by reference in its entirety for the specific purpose of disclosing various polycarbonate compositions and methods for manufacture of the same.

Generally polycarbonates can have a weight average molecular weight (Mw), of greater than about 5,000 g/mol based on PS standards. In one aspect, the polycarbonates can have an Mw of greater than or equal to about 20,000 g/mol, based on PS standards. In another aspect, the polycarbonates have an Mw based on PS standards of about 20,000 to 100,000 g/mol, including for example 30,000 g/mol, 40,000 g/mol, 50,000 g/mol, 60,000 g/mol, 70,000 g/mol, 80,000 g/mol, or 90,000 g/mol. In still further aspects, the polycarbonates have an Mw based on PS standards of about 22,000 to about 50,000 g/mol. In still further aspects, the polycarbonates have an Mw based on PS standards of about 25,000 to 40,000 g/mol.

In certain embodiments, the polycarbonate may comprise two or more polycarbonate compositions that differ in molecular weight and/or compositional variations.

Certain polycarbonates are sold under the trade name LEXAN™ by SABIC Innovative Plastics of Pittsfield, Mass.

### Pentaerythritol Tetrastearate

Pentaerythritol tetrastearate is used herein as a mold release agent that is useful in the molding of polycarbonate articles. In some embodiments, about 0.2 wt % to about 0.5 wt % of pentaerythritol tetrastearate is utilized. In other embodiments, about 0.25 wt % to about 0.35 wt % is utilized. This compound is commercially available from FACI Asia Pacific PTE LTD of Singapore and is of vegetable origin. The compound is depicted below.

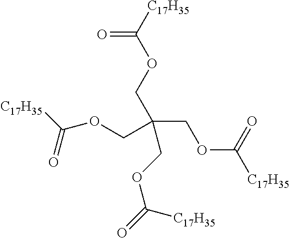

### Glycerol Monostearate

Glycerol monostearate is used herein as a mold release agent that is useful in the molding of polycarbonate articles. In some embodiments, about 0.05 wt % to about 0.3 wt % of glycerol monostearate is utilized. In other embodiments, the amount is about 0.5 wt % to about 0.15 wt %. The compound is commercially available and is depicted below.

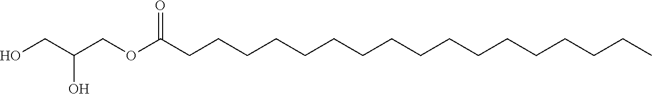 [SMILES: CCCCCCCCCCCCCCCCCC(=O)OCC(O)CO]

### Glycerol Tristearate

Glycerol tristearate is used herein as a mold release agent that is useful in the molding of polycarbonate articles. The compound is commercially available and is depicted below.

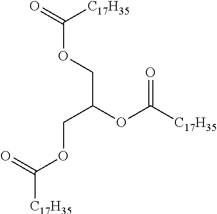

### Stabilizing Agents and Other Additives

Additional components can include an impact modifier, flow modifier, filler (e.g., a particulate polytetrafluoroethylene (PTFE), glass, carbon, mineral, or metal), reinforcing agent (e.g., glass fibers), antioxidant, heat stabilizer, light stabilizer, ultraviolet (UV) light stabilizer, UV absorbing additive, plasticizer, lubricant, release agent (such as a mold release agent), antistatic agent, anti-fog agent, antimicrobial agent, chain extender, colorant (e.g, a dye or pigment), de-molding agents, flow promoter, flow modifier, surface effect additive, radiation stabilizer, flame retardant, anti-drip agent (e.g., a PTFE-encapsulated styrene-acrylonitrile copolymer (TSAN)), or a combination comprising one or more of the foregoing.

Certain compositions comprise an antioxidant. Preferred antioxidants include phosphorous-containing compounds. Some preferred phosphorous-containing antioxidants are phosphite antioxidants. Examples of the phosphite antioxidants include tris(mixed mono- and di-nonylphenyl) phosphite, trisnonylphenyl phosphite, tris(2,4-di-t-butylphenyl) phosphite, priphenyl phosphite and diphenylmono-(2-ethylhexyl) phosphite, and examples of the phosphite compounds represented by the general formula (VI) include bis(2,4-di-t-butylphenyl)pentaerythritol diphosphite, bis(2,6-di-t-butyl-4-methylphenyl)pentaerythritol diphosphite and distearylpentaerythritol diphosphite. These phosphite compounds can be used singly or in a combination of two or more thereof. In some embodiments, a preferred antioxidant is tris(2,4-di-t-butylphenyl)phosphite.

### Molding

In one aspect, the present disclosure pertains to shaped, formed, or molded articles comprising the compositions described herein. Some preferred articles are components of medical devices such as syringes. Syringe components include the syringe housing, plunger, safety cap and other peripheral components.

The compositions can be molded into useful shaped articles by a variety of means such as injection molding, extrusion, rotational molding, blow molding and thermoforming to form articles. In some preferred processes, the article is injection molded. Such techniques are well known to those skilled in the art.

### Medical Devices

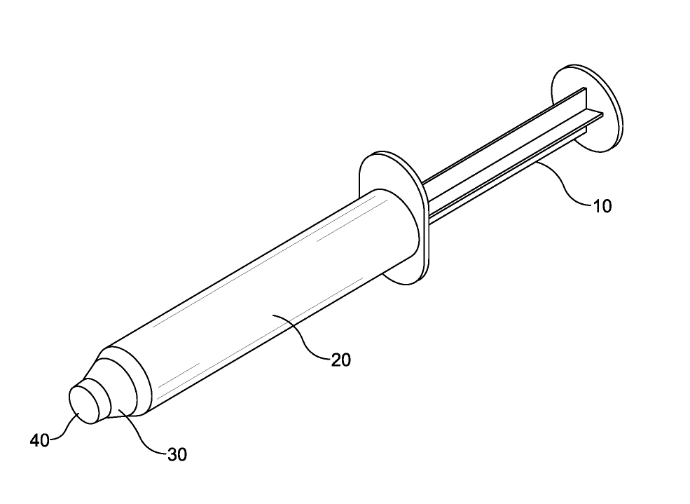

The instant disclosure is applicable to a wide variety of medical devices. Such devices include syringe parts such as syringe housing (also commonly referred to as the barrel), plunger, safety cap and other peripheral components. One representative syringe is presented in FIG. 1 showing a plunger 10, a barrel 20, a tip 30 and an optional safety cap 40. Other syringes may vary, for example, in shape, proportionality and optionally have additional features. Other medical devices include components of surgical instruments, cardiac surgery products, renal dialysis parts, blood collection and treatment systems, diagnostic equipment and connection components related to fluid transfer.

Processes known in the art for molding of polycarbonate-based medical devices are deficient in obtaining clean release from the mold—especially with product having a portion that is 4 mm or less in thickness. These known processes use a single mold release agent—typically agents derived from vegetables in order to comply with the regulations for plastics use in healthcare and medical device industries. The instant disclosure utilizes at least two mold release agents to overcome the mold release deficiencies.

### EXAMPLES

Table 1 and 2 list the detailed description of building blocks and the compositions of LEXAN™ polycarbonate-based formulations with veggie PETS mold release only (E1) versus a combination of dual mold release agents (E2, veggie PETS and GMS). The purpose of the use of mold release agent (PETS) that is derived from vegetables in the formulation (i.e., veggie PETS) is to comply with the regulations for plastics use in healthcare and medical device industries. The components of the examples shown here are compounded on a vacuum vented twin screw extruder using standard LEXAN™ extrusion condition.

TABLE 1
Detailed descriptions of the building blocks used in this disclosure

| # Item | Description | Supplier | Tradename |
|---|---|---|---|
| 1 PC1 | 100 grade polycarbonate homopolymer with an | SABIC-IP | Lexan ® ML4505-111 |
| | absolute Mw of 29,900 | | |
| 2 PC2 | High flow polycarbonate homopolymer with | SABIC-IP | Lexan ® ML5221-111 |
| | an absolute Mw of 21,900 | | |
| 3 STAB | Tris(2,4-di-t-butylphenyl)phosphite | CIBA/BASF | Irgafos 168 |
| 4 MR1 | Mold release, veggie pentaerythritol | LONZA INC | Glycolube PK |
| | tetrastearate | | |
| 5 MR2 | Mold release, glycerol monostearate | Mitsubishi | Rikemal S-100A |
| | | International | |

As shown in Table 2 the two examples differ only in the formulation by the use of single mold release agent, veggie PETS (0.5 phr) versus a combination of two mold release agents (PETS and GMS) (0.3 and 0.1 phr, respectively). The amounts of polycarbonate and stabilizer contents were kept constant to provide insights into the roles that single versus dual release agents play on release properties during part molding. With the formulation containing single mold release agent (E1), difficulty was experienced with the release of the syringe parts. In contrast, the use of dual mold release agents (veggie PETS and GMS) alleviated the above mentioned release difficulties, producing products with improved release from the mold.

In addition, the dual release concept can be applied to other aspects of healthcare related applications where part release property is crucial to the medical device manufacturing processes due to the complexity of part designs.

TABLE 2
Detailed compositions of experimental polycarbonate
formulations used for mold release study.

| # | Item | E1 | E2 |
|---|---|---|---|
| 1 | PC1 | 40 | 40 |
| 2 | PC2 | 60 | 60 |
| 3 | STAB | 0.03 | 0.03 |
| 4 | MR1 | 0.5 | 0.3 |
| 5 | MR2 | | 0.1 |

### Aspects

The present disclosure comprises at least the following aspects.

Aspect 1. A medical device part having a thickness of about 4 mm or less, the medical device part manufactured by an injection molding process utilizing a polycarbonate polymer and from about 0.2 wt % to about 0.5 wt % of pentaerythritol tetrastearate and from about 0.05 wt % to about 0.3 wt % of glycerol monostearate; wherein said pentaerythritol tetrastearate is derived from a biosource and all wt % values are based on the weight of the medical device part.

Aspect 2. The medical device part of Aspect 1 having a thickness of about 2 mm to about 4 mm.

Aspect 3. The medical device part of Aspect 1 having a thickness of about 2 mm to about 3 mm.

Aspect 4. The medical device part of any one of Aspects 1-3, wherein the polycarbonate polymer additionally comprises a stabilizing agent.

Aspect 5. The medical device part of Aspect 4, wherein the stabilizing agent comprises an antioxidant phosphite compound.

Aspect 6. The medical device part of Aspect 4, wherein the stabilizing agent comprises a phosphite-containing compound.

Aspect 7. The medical device part of any one of Aspects 1-6, wherein the medical device part is a component of a syringe.

Aspect 8. The medical device part of Aspect 7, wherein the component of the syringe is a syringe housing.

Aspect 9. The medical device part of anyone of Aspects 1-8, wherein the pentaerythritol tetrastearate is derived from one or more vegetable oils.

Aspect 10. A process for the manufacture of a medical device part, the process comprising injection molding polycarbonate polymer and comprising from about 0.2 wt % to about 0.5 wt % of pentaerythritol tetrastearate and from about 0.05 wt % to about 0.3 wt % of glycerol monostearate; wherein said pentaerythritol tetrastearate is derived from a biosource and all wt % values are based on the weight of the medical device part.

Aspect 11. The process of Aspect 10 having a thickness of about 2 mm to about 4 mm.

Aspect 12. The medical device part of Aspect 10 having a thickness of about 2 mm to about 3 mm.

Aspect 13. The process of any one of Aspects 10-12, wherein the polycarbonate polymer additionally comprises a stabilizing agent.

Aspect 14. The process of Aspect 13, wherein the stabilizing agent comprises an antioxidant phosphite compound.

Aspect 15. The process of Aspect 13, wherein the stabilizing agent comprises a phosphite-containing compound.

Aspect 16. The process of any one of Aspects 10-15, wherein the medical device part is a component of a syringe.

Aspect 17. The process of Aspect 16, wherein the component of the syringe is a syringe housing.

Aspect 18. The process of anyone of Aspects 10-17, wherein the pentaerythritol tetrastearate is derived from one or more vegetable oils.

Aspect 19. A medical device part having a thickness of about 4 mm or less, the medical device comprising a polycarbonate polymer, pentaerythritol tetrastearate and glycerol monostearate; wherein said pentaerythritol tetrastearate is derived from a biosource.

Aspect 20. The medical device of Aspect 19 additionally comprising a phosphite-containing compound.

Aspect 21. A medical device part having a thickness of about 4 mm or less, the medical device part manufactured by an injection molding process utilizing a polycarbonate polymer comprising pentaerythritol tetrastearate and one or both of glycerol monostearate and glycerol tristearate, wherein said pentaerythritol tetrastearate is derived from a biosource and the total amount of glycerol monostearate and glycerol tristearate does not exceed 1 wt % based on the weight of the medical device part.

Aspect 22. A medical device part having a thickness of about 4 mm or less, the medical device part manufactured by an injection molding process utilizing a polycarbonate polymer comprising pentaerythritol tetrastearate and full or partial stearate esters of glycerol, wherein said pentaerythritol tetrastearate is derived from a biosource and the total amount of the full or partial esters of stearate does not exceed 1 wt % based on the weight of the medical device part.

Aspect 23. A medical device part having a thickness of about 4 mm or less, the medical device part manufactured by an injection molding process utilizing a polycarbonate polymer comprising glycerol tristearate, wherein the total amount of glycerol tristearate does not exceed 1 wt % based on the weight of the medical device part.

### Definitions

It is to be understood that the terminology used herein is for the purpose of describing particular aspects only and is not intended to be limiting. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural equivalents unless the context clearly dictates otherwise. Thus, for example, reference to “a polycarbonate polymer” includes mixtures of two or more polycarbonate polymers.

As used herein, the term “combination” is inclusive of blends, mixtures, alloys, reaction products, and the like.

Ranges can be expressed herein as from one particular value to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±5% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Disclosed are the components to be used to prepare the compositions of the disclosure as hole as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

As used herein, the terms “weight average molecular weight” or “Mw” can be used interchangeably, and are defined by the formula:

$${M}_{w} = \frac{Σ {N}_{i} {M}_{i}^{2}}{Σ {N}_{i} {M}_{i}} ,$$

where Mi is the molecular weight of a chain and Ni is the number of chains of that molecular weight. Mw can be determined for polymers, e.g. polycarbonate polymers, by methods well known to a person having ordinary skill in the art using molecular weight standards, e.g. polycarbonate standards or polystyrene standards, preferably certified or traceable molecular weight standards.

As used herein, the term “biosource” indicates that the component is derived from a biological source rather than a petroleum-based process. Some components are derived from vegetable oils or other vegetable products.

By “full or partial stearate esters of glycerol” it is intended that a mixture of one or more of mono-, di- and tri-stearate esters of glycerol may be present.

The abbreviation “mm” represents millimeters. When used in terms of thickness, the measurement is at the thinnest portion of the article.

“Wt %” (or “wt %”) represents weight percent. Unless otherwise specified, wt % is based on the total weight of the composition.

The abbreviation “g” represents gram or grams.

“Mol” is the abbreviation for mole or moles.

The abbreviation “phr” means pounds per one hundred pounds. For example, 5 pounds of a component added to 100 pounds of polycarbonate is in an amount of 5 phr.

1. A medical device part having a thickness of about 4 mm or less, the medical device part manufactured by an injection molding process utilizing a polycarbonate polymer and from about 0.2 wt % to about 0.5 wt % of pentaerythritol tetrastearate and from about 0.05 wt % to about 0.3 wt % of glycerol monostearate; wherein said pentaerythritol tetrastearate is derived from a biosource and all wt % values are based on the weight of the medical device part.

2. The medical device part of claim 1 having a thickness of about 2 mm to about 4 mm.

3. The medical device part of claim 1 having a thickness of about 2 mm to about 3 mm.

4. The medical device part of claim 1, wherein the polycarbonate polymer additionally comprises a stabilizing agent.

5. The medical device part of claim 4, wherein the stabilizing agent comprises an antioxidant phosphite compound.

6. The medical device part of claim 4, wherein the stabilizing agent comprises a phosphite-containing compound.

7. The medical device part of claim 1, wherein the medical device part is a component of a syringe.

8. The medical device part of claim 7, wherein the component of the syringe is a syringe housing.

9. The medical device part of claim 1, wherein the pentaerythritol tetrastearate is derived from one or more vegetable oils.

10. A process for the manufacture of a medical device part, the process comprising injection molding polycarbonate polymer and comprising from about 0.2 wt % to about 0.5 wt % of pentaerythritol tetrastearate and from about 0.05 wt % to about 0.3 wt % of glycerol monostearate; wherein said pentaerythritol tetrastearate is derived from a biosource and all wt % values are based on the weight of the medical device part.

11. The process of claim 10 having a thickness of about 2 mm to about 4 mm.

12. The medical device part of claim 10 having a thickness of about 2 mm to about 3 mm.

13. The process of claim 10, wherein the polycarbonate polymer additionally comprises a stabilizing agent.

14. The process of claim 13, wherein the stabilizing agent comprises an antioxidant.

15. The process of claim 13, wherein the stabilizing agent comprises a phosphite-containing compound.

16. The process of claim 10, wherein the medical device part is a component of a syringe.

17. The process of claim 16, wherein the component of the syringe is a syringe housing.

18. The process of claim 10, wherein the pentaerythritol tetrastearate is derived from one or more vegetable oils.

19. A medical device part having a thickness of about 4 mm or less, the medical device comprising a polycarbonate polymer, pentaerythritol tetrastearate and glycerol monostearate; wherein said pentaerythritol tetrastearate is derived from a biosource.

20. The medical device of claim 19 additionally comprising a phosphite-containing compound.
